(19) United States
(12) Patent Application Publication
(10) Pub. No.: US 2004/0203030 A1
(43) Pub. Date: Oct. 14, 2004

(54) Alternatively spliced isoform of human COX1

(76) Inventor: Philip W. Garrett-Engele, Seattle, WA (US)

Correspondence Address: R. Douglas Bradley, Merck & Co., Inc., Patent Department RY60-30, P.O. Box 2000, Rahway, NJ, 07065-0907, US

(21) Appl. No.: 10/741,853

(22) Filed: Dec. 18, 2003

Related U.S. Application Data
(60) Provisional application No. 60/435,478, filed on Dec. 19, 2002.

(51) Int. Cl. C12Q 1/68; G01N 33/53; C07H 21/04; C12N 9/02

(52) U.S. Cl. 435/6; 435/7.1; 435/69.1; 435/189; 435/320.1; 435/325; 536/23.2

(57) ABSTRACT

The present invention features nucleic acids and polypeptides encoding one novel splice variant isoform of COX1. The polynucleotide sequence of COX1sv1 is provided by SEQ ID NO 1. The amino acid sequence for COX1sv1 is provided by SEQ ID NO 2. The present invention also provides methods for using COX1sv1 polynucleotides and proteins to screen for compounds that bind to COX1sv1.

This application claims priority to U.S. Provisional Patent Application Serial No. 06/435,478 filed on Dec. 19, 2002, which is incorporated by reference herein in its entirety.

### BACKGROUND OF THE INVENTION

The references cited herein are not admitted to be prior art to the claimed invention.

Prostaglandins are important autocrine and paracrine lipid mediators involved in the diverse processes of cell proliferation, inflammatory and immune responses, smooth muscle contraction, maintenance of fluid and electrolyte balance, platelet aggregation, and the production of extracellular matrix proteins (Zurier, 1990 Adv. Prostaglandin Thromboxaned Leukotriene Res., 21:947-954; Varga et al., 1987 Biochem. Biophys. Res. Commun., 147:1282-1288; Needleman, et al,. 1986 Annu. Rev. Biochem., 55:69-102). Prostaglandins are synthesized de novo from membrane-released arachidonic acid when cells are activated by mechanical trauma or by specific cytokine, growth factor, or other stimulus (Funk, 2001 Science, 294:1871-1875).

Prostaglandin-endoperoxide H synthases (PTGS or PGH), also known as cyclooxygenases (COX), are responsible for catalyzing the committed step in the conversion of arachidonic acid to prostaglandins (Smith et al. 2000 Annu. Rev. Biochem., 69:145-182; Prescott and Yost, 2002 Proc. Natl. Acad. Sci., 99:9084-9086). The products of the prostanoid pathway are involved in inflammatory conditions, especially in chronic inflammatory diseases such as rheumatoid arthritis (Harris, 1990 N. Engl. J. Med,. 322:1277-1289). Analysis of arthritic patients and animal models of rheumatoid arthritis demonstrate that the intensity of COX expression directly correlates with disease severity (Sano et al., 1992 J. Clin. Invest., 89:97-108). Prostaglandin-endoperoxide H synthases exists as two isoforms, COX1 (also known as PTGS1 or PGH1) and COX2 (also known as PTGS2 or PGH2). Generally, COX1 is the constitutively expressed isoform, while COX2 is the isoform that is induced by growth factors, hormones and cytokines (Funk, 2001 Science, 294:1871-1875; Vogiagis et al., 2001 Carcinogenesis, 22:869-874; Appleby et al., 1994 Biochem. J., 302:723-727). COX1 is thought to be responsible, in large part, for endogenous basal release of prostaglandins and hence is important in their physiological functions such as the maintenance of gastrointestinal integrity and renal blood flow. In contrast, COX2 is thought to be mainly responsible for the pathological effects of prostaglandins where rapid induction of the enzyme occurs in response to such agents as inflammatory agents, hormones, growth factors, and cytokines (see WO 94/14977).

The COX1 gene on chromosome 9 spans 40 kilobases and contains 11 exons. The COX2 gene, located on chromosome 1, is more than 8.3 kilobases long and contains 10 exons (Kosaka et al., 1994 Eur. J. Biochem., 221:889-897). The COX1 and COX2 polypeptides share about 61% amino acid sequence identity (Appleby et al., 1994 Biochem. J., 302:723-727). One alternatively spliced isoform of COX1 has been described and results in the alternative splicing of exon 9 which eliminates 111 base pairs (bps) of coding sequence, including one potential glycosylation site (Diaz et al., 1992 J. Biol. Chem., 267:10816-10822).

Non-steroidal anti-inflammatory drugs (NSAIDs), such as aspirin and ibuprofen, are known to block COX-derived prostaglandin synthesis and thus are commonly used as analgesics and anti-inflammatory drugs (Funk, 2001 Science, 294:1871-1875). Aspirin's mechanism of action on COX1 and COX2 is unique in that it covalently acetylates a serine residue in both proteins and blocks substrate access and orientation at the active site. Therefore, aspirin affects prostaglandin synthesis through both COX1 and COX2. By contrast, the coxibs class of COX inhibitors (including refocoxib and celecoxib) are newer COX2-specific drugs that have been used clinically for the treatment of arthritis and other pain management (FitzGerald et al., 2001 N. Engl. J. Med. 345:433).

While nonselective NSAIDs (including aspirin) have been associated with gastroduodenal mucosal injury, such as ulcers and ulcer complications, selective COX2 inhibitors have demonstrated improved gastrointestinal safety and tolerability (Konstam et al., 2001 Circulation, 104:2280-2288). Clinical use of coxibs has demonstrated that they are as effective as traditional nonselective NSAIDs and also reveal a 50% reduction in adverse gastrointestinal events (FitzGerald et al., 2001 N. Engl. J. Med. 345:433). Thus, the increased rate of gastrointestinal mucosal injury results from inhibition of COX1 (Goldstein et al., 2000 American Journal of Gastroenterology, 95:1681-1690).

The use of NSAIDs, and in particular aspirin, has been extended to prophylaxis of cardiovascular disease (Halter et al., 2001 Gut, 49:443-453). COX1 inhibition has been associated with decreased synthesis of platelet-derived thrombioxane, a vasoconstrictor and potent inducer of platelet aggregation (Catella-Lawson and Crofford, 2001 Am. J. Med., 110:28S-32S). The sustained inhibition of COX1-mediated thromboxane synthesis underlies the efficacy of aspirin in significantly reducing the incidence of cardiovascular death, myocardial infarction, and stroke in high-risk patients (Konstam et al., 2001 Circulation, 104:2280-2288).

Long-term treatment with NSAIDs in both clinical and animal studies also reduces the incidence of colorectal cancer by 40-60%. (Vogiagis et al., 2001 Carcinogenesis, 22: 869-874). Furthermore, increased expression of COX2 m-RNA has been reported in human colorectal cancer and in carcinogen induced rodent models of colon cancer. The disruption of the COX2 gene in such mice significantly reduces the number of tumors formed. These results suggest that COX2 plays an important role in the early adenoma formation (Vogiagis et al., 2001 Carcinogenesis, 22:869-874). In contrast, COX1 mRNA expression is not generally altered in colorectal cancer. A splice variant of the rat COX1 gene has also been detected in colon cancer (Vogiagis et al., 2001 Carcinogenesis, 22:869-874). COX2 is also expressed in prostate carcinoma (Yoshimura et al., 2000 Cancer, 89:589-596). While the precise mechanism of the action for the chemoprotective effect of NSAIDs is unknown, it now appears that COX activity is directly involved (Staats, 2002 Journal of Pain and Symptom Management, 24:S4-S9). Thus coxibs, through their selective action on COX2, may have utility in treating and possibly preventing cancer (Staats, 2002 Journal of Pain and Symptom Management, 24:S4-S9).

Investigators have also demonstrated a link between COX and Alzheimer's disease. These studies revealed that the relative risk of Alzheimer's was significantly lower in patients with a history of NSAID use, but not among those who had been using aspirin or acetaminophen thereby suggesting a link between Alzheimer's and COX2 activity (Stewart et al., 1997 Neurology, 48:626-632; Andersen et al., 1995 Neurology, 45:1441-1445).

Thus, while some compounds have been identified that alter COX1 and COX2 activity to achieve a therapeutic benefit, there still remains a substantial need in the art for additional compounds that inhibit COX1 and COX2. Because of the importance of COX1 and COX2 as drug targets and their roles in inflammation, pain management, cancer, heart disease, and possibly Alzheimer's disease, there is a need in the art for COX1 and COX2 polynucleotides and proteins and methods of use thereof that can be used to identify compounds that selectively bind to isoforms of human COX1 and COX2. The present invention is directed towards a novel COX1 isoform and uses thereof.

### SUMMARY OF THE INVENTION

RT-PCR has been used to identify and confirm the presence of a novel human splice variant of COX1 mRNA. More specifically, the present invention features the polynucleotide encoding one protein isoform of COX1. The polynucleotide sequence encoding COX1sv1 is provided by SEQ ID NO 1. The amino acid sequence for COX1sv1 is provided by SEQ ID NO2.

Thus, a first aspect of the present invention describes a purified COX1sv1 encoding nucleic acid. The COX1sv1 encoding nucleic acid comprises SEQ ID NO 1 or the complement thereof. Reference to the presence of one region does not indicate that another region is not present. For example, in different embodiments the inventive nucleic acid can comprise, consist, or consist essentially of a nucleic acid encoding for SEQ ID NO 1.

Another aspect of the present invention describes a purified COX1sv1 polypeptide that can comprise, consist or consist essentially of the amino acid sequence of SEQ ID NO2.

Another aspect of the present invention describes two expression vectors. In one embodiment of the invention, the inventive expression vector comprises a nucleotide sequence encoding a polypeptide comprising, consisting, or consisting essentially of SEQ ID NO 2, wherein the nucleotide sequence is transcriptionally coupled to an exogenous promoter. Alternatively, the nucleotide sequence comprises, consists, or consists essentially of SEQ ID NO 1, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention, describes a recombinant cell made by a process comprising the step of introducing into the cell an expression vector comprising a nucleotide sequence comprising, consisting, or consisting essentially of SEQ ID NO 1 or a nucleotide sequence encoding a polypeptide comprising, consisting, or consisting essentially of an amino acid sequence of SEQ ID NO 2, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing COX1sv1 polypeptide comprising SEQ ID NO 2. The method involves the step of growing a recombinant cell containing an inventive expression vector under conditions wherein the polypeptide is expressed from the expression vector.

Another aspect of the present invention features a purified antibody preparation comprising an antibody that binds selectively to COX1sv1 as compared to one or more COX1 isoform polypeptides that are not COX1sv1.

Another aspect of the present invention provides a method of screening for a compound that binds to COX1sv1, or fragments thereof. In one embodiment, the method comprises the steps of: (a) expressing a polypeptide comprising the amino acid sequence of SEQ ID NO 2 or a fragment thereof from recombinant nucleic acid; (b) providing to said polypeptide a test preparation comprising one or more test compounds; and measuring the level of binding of, said test preparation to said polypeptide comprising SEQ ID NO 2.

In another embodiment of the method, a compound is identified that binds selectively to COX1sv1 polypeptide as compared to one or more COX1 or COX2 isoform polypeptides that are not COX1sv1. This method comprises the steps of: (a) providing a COX1sv1 polypeptide comprising SEQ ID NO 2; (b) providing a COX1 or COX2 isoform polypeptide that is not COX1sv1, (c) contacting said COX1sv1 polypeptide and said COX1 or COX2 isoform polypeptide that is not COX1sv1 with a test preparation comprising one or more test compounds; and (d) determining the binding of said test preparation to said COX1sv1 polypeptide and to said COX1 or COX2 isoform polypeptide that is not COX1sv1, wherein a test preparation that binds to said COX1sv1 polypeptide but does not bind to said COX1 or COX2 isoform polypeptide that is not COX1sv1 contains a compound that selectively binds said COX1sv1 polypeptide.

In another embodiment of the invention, a method is provided for screening for a compound able to bind to or interact with a COX1sv1 protein or a fragment thereof comprising the steps of: (a) expressing a COX1sv1 polypeptide comprising SEQ ID NO 2 or a fragment thereof from a recombinant nucleic acid; (b) providing to said polypeptide a labeled COX1 ligand that binds to said polypeptide and a test preparation comprising one or more compounds; and (c) measuring the effect of said test preparation on binding of said labeled COX1 ligand to said polypeptide, wherein a test preparation that alters the binding of said labeled COX1 ligand to said polypeptide contains a compound that binds to or interacts with said polypeptide.

In yet another embodiment of the invention, a method is provided of screening for COX1sv1 activity comprising the steps of: (a) contacting a cell expressing a recombinant nucleic acid encoding COX1sv1 comprising SEQ ID NO 2 with a test preparation comprising one or more test compounds; and (b) measuring the effect of said test preparation on one or more functional properties of COX1.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE FIGURES

FIG. 1A illustrates the exon structure of COX1 mRNA corresponding to the known long reference form of COX1 mRNA (labeled NM_{—}080591) and the inventive short form splice variants of COX1 mRNA (labeled COX1sv1). The small arrows above exons 4 and 7 show the positions of the oligonucleotide primers used to perform RT-PCR assays to confirm the exon structure of COX1 mRNA in the 80 samples (see Example 1). FIG. 1B shows the nucleotide sequences of the COX1 and COX1sv1 mRNAs exon junctions that results from splicing of exon 4 to exon 5 [SEQ ID NO 3] and exon 5 to exon 6 [SEQ ID NO 4] in the case of the COX1 mRNA, and the splicing of exon 4 to exon 6 [SEQ ID NO 5] in the case of COX1sv1 mRNA. For the exon 4 to exon 5 splice junction, the nucleotide shown in italics represent the 20 nucleotides at the 3′ end of exon 4 and nucleotides shown in boldface font represent the 20 nucleotides at the 5′ end of exon 5. For the exon 5 to exon 6 splice junction, the nucleotide shown in boldface font represent the 20 nucleotides at the 3′ end of exon 5 and nucleotides shown in underline represent the 20 nucleotides at the 5′ end of exon 6. For the exon 4 to exon 6 splice junction found in the inventive COXsv1 mRNA, the nucleotides in italics represent the 20 nucleotides at the 3′ end of exon 4, while the nucleotides in underline represent the 20 nucleotides at the 5′ end of exon 6.

### DEFINITIONS

Unless defined otherwise, all technical and scientific terms used herein have the meaning commonly understood by one of ordinary skill in the art to which this invention belongs.

As used herein, “COX1” refers to the cyclooxygenase 1 protein (NP_{—}542158). In contrast, reference to a COX1 isoform, includes NP_{—}542158 and other polypeptide isoform variants of COX1.

As used herein, “COX1sv1” refers to a splice variant isoform of human COX1 protein having an amino acid sequence set forth in SEQ ID NO 2.

As used herein, “COX1” refers to polynucleotides encoding COX1.

As used herein, “COX1sv1” refers to polynucleotides encoding COX1sv1 having an amino acid sequence set forth in SEQ ID NO 2.

As used herein, “COX2” refers to the cyclooxygenase 2 protein (NP_{—}000954).

As used herein, “COX2” refers to polynucleotides, such as NM_{—}000963, that encode COX1.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

The present invention relates to the nucleic acid sequence encoding human COX1sv1, an alternatively spliced isoform of COX1, and to the amino acid sequences encoding this protein. SEQ ID NO 1 is the polynucleotide sequence representing the open reading frame that encodes the COX1sv1 proteins. SEQ ID NO 2 shows the polypeptide sequence of COX1sv1.

COX1sv1 polynucleotide sequence encoding COX1sv1 proteins, as exemplified and enabled herein include a number of specific, substantial and credible utilities. For example, COX1sv1 encoding nucleic acids were identified in a mRNA sample obtained from a human source (see Example 1-3). Such nucleic acids can be used as hybridization probes to distinguish between cells that produce COX1sv1 transcripts from human or non-human cells (including bacteria) that do not produce such transcripts. Similarly, antibodies specific for COX1sv1 can be used to distinguish between cells that express COX1sv1 from human or non-human cells (including bacteria) that do not express COX1sv1.

COX1 is an important drug target for compounds that have therapeutic value in the management of pain, inflammation, heart disease, and blood coagulation. For example, a large number of non-steroidal anti-inflammatory drugs are known that bind to and inhibit COX1 and COX2. One well known NSAID is aspirin, which irreversibly inhibitsCOX1 enzyme activity by covalently acetylating a serine residue, thereby blocking proper substrate access and orientation at the active site (Funk, 2001, Science 294:1871-1875). COX Given the importance of COX1 and COX2 activity to the therapeutic management of inflammation and, in the case of COX1 activity in platelets, as a prophylactic for cardiovascular disease, it is of value to identify COX1 isoforms and identify COX1-ligand compounds that are isoform-specific as well as compounds that are effective ligands for COX1 and/or COX2 isoforms. In particular, it may be important to identify compounds that are effective inhibitors of a specific COX1 isoform activity, yet do not bind to a plurality of other COX1 and/or COX2 isoforms. Compounds that bind to multiple COX1 and/or COX2 isoforms may require higher drug doses to saturate multiple COX1 isoform-binding sites, and thereby result in a greater likelihood of secondary non-therapeutic side effects. For the foregoing reasons, the COX1sv1 COX protein represents a useful compound binding target and has utility in the identification of new COX1 and/or COX2 ligands exhibiting a preferred specificity profile and having greater efficacy for their intended use.

In some embodiments, COX1sv1 activity is modulated by a ligand compound to achieve one or more of the following: manage pain due to inflammation, particularly osteoarthritis and rheumatoid arthritis, and as a prophylaxis of cardiovascular disease (Hennekens, 1999, Am. Heart J. 137:S9-13). Compounds that treat inflammation and cardiovascular disease are particularly important because of the large number of individuals that benefit from use of such therapeutic compounds (For a review of NSAIDs and the treatment of inflammation, see: Roberts and Morrow, In, Goodman & Gilman's The Pharmacological Basis of Therapeutics, 10th Ed., McGraw-Hill, New York, 2001, Ch. 27, pp. 687-731).

Compounds modulating COX1sv1 include agonists, antagonists, and allosteric modulators. Generally, but not always, COX1sv1-antagonists and allosteric modulators negatively affecting COX1sv1 activity will be used to inhibit COX1 activity thereby decreasing prostaglandin synthesis. Inhibitors of COX1 achieve clinical efficacy by a number of effects, including inhibition of prostaglandin biosynthesis, including thromboxane A₂, an inducer of platelet aggregation that results in decreased risk of myocardial infarction, stroke and peripheral vascular thromboses.

COX1sv1 activity can also be affected by modulating the cellular abundance of transcripts encoding COX1sv1. Compounds modulating the abundance of transcripts encoding COX1sv1 include a cloned polynucleotide encoding COX1sv1 that can express COX1sv1 in vivo, antisense nucleic acids targeted to COX1sv1 transcripts, and enzymatic nucleic acids, such as ribozymes and RNAi, targeted to COX1sv1 transcripts.

In some embodiments, COX1sv1 activity is modulated to achieve a therapeutic effect upon diseases. For example, the risk of myocardial infarction, stroke and peripheral vascular thromboses may be lowered by modulating COX1sv1 activity to achieve, for instance, decreased levels of prostaglandins, in particular thromboxane A₂.

COX1sv1 NUCLEIC ACID

COX1sv1 nucleic acids contain regions that encode for polypeptides comprising, consisting, or consisting essentially of SEQ ID NO 2. COX1sv1 nucleic acids have a variety of uses, such as being used as a hybridization probe or PCR primer to identify the presence of COX1sv1 nucleic acid; being used as a hybridization probe or PCR primer to identify nucleic acid encoding for proteins related to COX1sv1 and/or being used for recombinant expression of COX1sv1 polypeptides. In particular, COX1sv1 polynucleotides do not have the polynucleotide regions that comprise exon 5 of the COX1 gene.

Regions in COX1sv1 nucleic acid that do not encode for COX1sv1 or are not found in SEQ ID NO 1 if present, are preferably chosen to achieve a particular purpose. Examples of additional regions that can be used to achieve a particular purpose include capture regions that can be used as part of an ELISA sandwich assay, reporter regions that can be probed to indicate the presence of the nucleic acid, expression vector regions, and regions encoding for other polypeptides.

The guidance provided in the present application can be used to obtain the nucleic acid sequence encoding COX1sv1 related proteins from different sources. Obtaining nucleic acids encoding COX1sv1 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

COX1sv1 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

COX1sv1 Probes

Probes for COX1sv1 contain a region that can specifically hybridize to COX1sv1 target nucleic acids, under appropriate hybridization conditions and can distinguish COX1sv1 nucleic acid from non-target nucleic acids, in particular COX1 polynucleotides containing exon 5. Probes for COX1sv1 can also contain nucleic acid regions that are not complementary COX1sv1 nucleic acids.

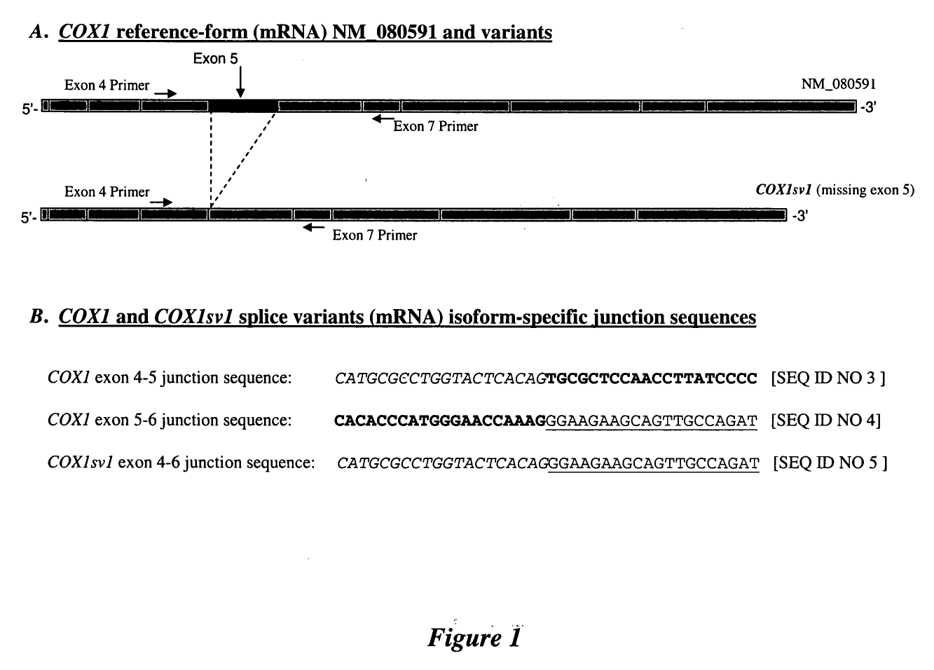

In embodiments where, for example, COX1sv1 polynucleotide probes are used in hybridization assays to specifically detect the presence of COX1sv1 polynucleotides in samples, the COX1sv1 polynucleotides comprise at least 20 nucleotides of the COX1sv1 sequence that corresponds to the novel exon junction polynucleotide regions. In particular, for detection of COX1sv1, the probe comprises at least 20 nucleotides of the COX1sv1 sequence that corresponds to an exon junction polynucleotide created by the alternative splicing of exon 4 to exon 6 of the primary transcript of the reference COX1 gene (see FIG. 1B). For example, the polynucleotide sequence: 5′ GTACTCACAGGGAAGAAGCA 3′ [SEQ ID NO 6] represents one embodiment of such an inventive COX1sv1 polynucleotide wherein a first 10 nucleotide region is complementary and hybridizable to the 3′ end of exon 4 of the COX1 gene and a second 10 nucleotide region is complementary and hybridizable to the 5′ end of exon 6 of the COX1 gene (see FIG. 1B).

In some embodiments, the first 20 nucleotides COX1sv1 comprises a first continuous region of 5 to 15 nucleotides that is complementary and hybridizable to the 3′ end of exon 4 and a second continuous region of 5 to 15 nucleotides that is complementary and hybridizable to the 5′ end exon 6.

In other embodiments, the COX1sv1 polynucleotide comprises at least 40, 60, 80 or 100 nucleotides of the COX1sv1 sequence that corresponds to a junction polynucleotide region created by the alternative splicing of exon 4 to exon 6 in the case of COX1sv1 of the primary transcript of the COX1 gene. In embodiments involving COX1sv1, the COX1sv1 polynucleotide is selected to comprise a first continuous region of at least 5 to 15 nucleotides that is complementary and hybridizable to the 3′ end of exon 26 and a second continuous region of at least 5 to 15 nucleotides that is complementary and hybridizable to the 5′ end of exon 30. As will be apparent to a person of skill in the art, a large number of different polynucleotide sequences from the region of the exon 4 to exon 6 splice junction may be selected which will, under appropriate hybridization conditions, have the capacity to detectably hybridize to COX1sv1 polynucleotides, and yet will hybridize to a much less extent or not at all to COX1 isoform polynucleotides wherein exon 4 is not spliced to exon 6.

Preferably, non-complementary nucleic acid that is present has a particular purpose such as being a reporter sequence or being a capture sequence. However, additional nucleic acid need not have a particular purpose as long as the additional nucleic acid does not prevent the COX1sv1 nucleic acid from distinguishing between target polynucleotides, e.g., COX1sv1 polynucleotides and non-target polynucleotides, including, but not limited to COX1 polynucleotides not comprising the exon 4 to exon 6 splice junction.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tm the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

COX1sv1 polynucleotides, such as those comprising SEQ ID NO 1 can be used to make COX1sv1 polypeptides. In particular, COX1sv1 polypeptides can be expressed from recombinant nucleic acid in a suitable host or in vitro using a translation system. Recombinantly expressed COX1sv1 polypeptides can be used, for example, in assays to screen for compounds that bind to COX1sv1. Alternatively, COX1sv1 polypeptides can also be used to screen for compounds that bind to one or more COX1 and/or COX2 isoforms but do not bind to COX1sv1.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLac1 (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460), and. Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171).

To enhance expression in a particular host it may be useful to modify the sequence provided in SEQ ID NO 1 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acid encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

COX1sv1 Polypeptides

COX1sv1 polypeptides contain an amino acid sequence comprising, consisting, or consisting essentially of SEQ ID NO 2. COX1sv1 polypeptides have a variety of uses, such as providing a marker for the presence of COX1sv1; being used as an immunogen to produce antibodies binding to COX1sv1, respectively; being used as a target to identify compounds binding selectively to COX1sv1; or being used in an assay to identify compounds that bind to one or more isoforms of COX1 and/or COX2 but do not bind to or interact with COX1sv1.

In chimeric polypeptides containing one or more regions from COX1sv1 and one or more regions not from COX1sv1 the region(s) not from COX1sv1, can be used, for example, to achieve a particular purpose or to produce a polypeptide that can substitute for COX1sv1 or fragments thereof. Particular purposes that can be achieved using chimeric COX1sv1 polypeptides include providing a marker for COX1sv1 activity, enhancing an immune response, and modulating prostaglandin levels.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional COX1sv1

Functional COX1sv1 is a different protein isoform-of COX1. The identification of the amino acid and nucleic acid sequences of COX1sv1 provides tools for obtaining functional proteins related to COX1sv1 from other sources, for producing COX1sv1 chimeric proteins, and for producing functional derivatives of SEQ ID NO 2.

COX1sv1 polypeptides can be readily identified and obtained based on their sequence similarity to COX1sv1 (SEQ ID NO 2). In particular, COX1sv1 polypeptides lack the amino acids coded by exon 5 of the COX1 gene. Both the amino acid and nucleic acid sequences of COX1sv1 can be used to help identify and obtain COX1sv1 polypeptides. For example, SEQ ID NO 1 can be used to produce degenerative nucleic acid probes or primers for identifying and cloning nucleic acid polynucleotides encoding for a COX1sv1 polypeptide. In addition, polynucleotides comprising, consisting, or consisting essentially of SEQ ID NO 1 or fragments thereof, can be used under conditions of moderate stringency to identify and clone nucleic acid encoding COX1sv1 polypeptides from a variety of different organisms. The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with COX1sv1 obtained from a particular source, derivatives can be produced. Such derivatives include polypeptides with amino acid substitutions, additions and deletions. Changes to COX1sv1 to produce a derivative having essentially the same properties should be made in a manner not altering the tertiary structure of COX1sv1.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acidto be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

COX1sv1 Antibodies

Antibodies recognizing COX1sv1 can be produced using a polypeptide containing SEQ ID NO 2, or a fragment thereof as an immunogen. Preferably, a COX1sv1 polypeptide used as an immunogen consists of a polypeptide of SEQ ID NO 2 or a SEQ ID NO 2 fragment having at least 10 contiguous amino acids in length corresponding to the polynucleotide region representing the junction resulting from the splicing of exon 4 to exon 6 of the COX1 gene.

In some embodiments where, for example, COX1 sv1 polypeptides are used to develop antibodies that bind specifically to COX1sv1 and not to other isoforms of COX1 or COX2, the COX1sv1 polypeptides comprise at least 10 amino acids of the COX1sv1 polypeptide sequence corresponding to a junction polynucleotide region created by the alternative splicing of exon 4 to exon 6 of the primary transcript the COX1 gene (see FIG. 1). For example, the amino acid sequence: amino terminus-RLVLTGKKQL-carboxy terminus [SEQ ID NO 7], represents one embodiment of such an inventive COX1sv1 polypeptide wherein a first 5 amino acid region is encoded by nucleotide sequence at the 3′ end of exon 4 of the COX1 gene and a second 5 amino acid region is encoded by the nucleotide sequence directly after the novel splice junction. Preferably, at least 10 amino acids of the COX1sv1 polypeptide comprises a first continuous region of 2 to 8 amino acids that is coded by nucleotides at the 3′ end of exon 4 and a second continuous region of 2 to 8 amino acids that is coded by nucleotides at the 5′ end exon 6.

In other embodiments, COX1sv1-specific antibodies are made using a COX1sv1 polypeptide that comprises at least 20, 30, 40 or 50 amino acids of the COX1sv1 sequence that corresponds to a junction polynucleotide region created by the alternative splicing of exon 4 to exon 6 of the primary transcript of the COX1 gene. In each case the COX1sv1 polypeptides are selected to comprise a first continuous region of at least 5 to 15 amino acids that is coded by nucleotides at the 3′ end of exon 4 and a second continuous region of 5 to 15 amino acids that is coded by nucleotides directly after the novel splice junction.

Antibodies to COX1sv1 have different uses such as being used to identify the presence of COX1sv1, and to isolate COX1sv1 polypeptides. Identifying the presence of COX1sv1 can be used, for example, to identify cells producing COX1sv1. Such identification provides an additional source of COX1sv1 and can be used to distinguish cells known to produce COX1sv1 from cells that do not produce COX1sv1. For example, antibodies to COX1sv1 can distinguish human cells expressing COX1sv1 from human cells not expressing COX1sv1 or non-human cells (including bacteria) that do not express COX1sv1. Such COX1sv1 antibodies can also be used to determine the effectiveness of COX1sv1 ligands, using techniques well known in the art, to detect and quantify changes in the protein levels of COX1sv1 in cellular extracts, and in situ immunostaining of cells and tissues.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

COX1sv1 Binding Assay

COX1sv1 or fragments thereof can be used in binding studies to identify compounds binding to or interacting with COX1sv1 or fragments thereof. In one embodiment, the COX1sv1 or a fragment thereof can be used in binding studies with COX1 or COX2 isoform proteins or a fragment thereof, to identify compounds that: bind to or interact with COX1sv1 and other COX1 or COX2 isoforms; bind to or interact with one or more other COX1 and COX2 isoforms and not with COX1sv1. Such binding studies can be performed using different formats including competitive and non-competitive formats. Further competition studies can be carried out using additional compounds determined to bind to COX1sv1, or other COX1 and/or COX2 isoforms.

The particular COX1sv1 sequence involved in ligand binding can be readily identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using COX1sv1 expressed from a recombinant nucleic acid. Alternatively, recombinantly expressed COX1sv1 consists of the SEQ ID NO 2 amino acid sequence.

Binding assays can be performed using individual compounds or preparations containing different numbers of compounds. A preparation containing different numbers of compounds having the ability to bind to COX1sv1 can be divided into smaller groups of compounds that can be tested to identify the compound(s) binding to COX1sv1.

Binding assays can be performed using recombinantly produced COX1sv1 present in different environments. Such environments include, for example, cell extracts and purified cell extracts containing a COX1sv1 recombinant nucleic acid; and also include, for example, the use of a purified COX1sv1 polypeptide produced by recombinant means which is introduced into different environments.

In one embodiment of the invention, a binding method is provided for screening for a compound able to bind selectively to COX1sv1. The method comprises the steps: providing a COX1sv1 polypeptide comprising SEQ ID NO 2; providing a COX1 or COX2 isoform polypeptide that is not COX1sv1, contacting the COX1sv1 polypeptide and the COX1 or COX2 isoform polypeptide that is not COX1sv1 with a test preparation comprising one or more test compounds; and then determining the binding of the test preparation to the COX1sv1 polypeptide and to the COX1 or COX2 isoform polypeptide that is not COX1sv1 wherein a compound which binds to the COX1sv1 polypeptide but does not bind to COX1 or COX2 isoform polypeptide that is not COX1sv1 contains one or more compounds that selectively binds to COX1sv1.

In another embodiment of the invention, a binding method is provided for screening for a compound able to bind selectively to a COX1 or COX2 isoform polypeptide that is not COX1sv1. The method comprises the steps: providing a COX1sv1 polypeptide comprising SEQ ID NO 2; providing a COX1 or COX2 isoform polypeptide that is not COX1sv1, contacting the COX1sv1 polypeptide and the COX1 or COX2 isoform polypeptide that is not COX1sv1 with a test preparation comprising one or more test compounds; and then determining the binding of the test preparation to the COX1sv1 polypeptide and the COX1 or COX2 isoform polypeptide that is not-COX1sv1, wherein a test preparation that binds the COX1 or COX2 isoform polypeptide that is not COX1sv1 but does not bind the COX1sv1 contains a compound that selectively binds the COX1 isoform polypeptide that is not COX1sv1.

The above-described selective binding assays can also be performed with a polypeptide fragment of COX1sv1, wherein the polypeptide fragment comprises at least 10 consecutive amino acids that are coded by a nucleotide sequence that bridges the junction created by the splicing of the 3′ end of exon 4 to the 5′ end of exon 6 in the case of COX1sv1. Similarly, the selective binding assays may also be performed using a polypeptide fragment of a COX1 or COX2 isoform polypeptide that is not COX1sv1 wherein the polypeptide fragment comprises at least 10 consecutive amino acids that are coded by: a) a nucleotide sequence that is contained within exon 5 of the COX1 gene; or b) a nucleotide sequence that bridges the junction created by the splicing of the 3′ end of exon 4 to the 5′ end of exon 5, the splicing of the 3′ end of exon 5 to the 5′ end of exon 6 of the COX1 gene, or c) a nucleotide sequence encoding COX2, such as NM_{—}000963.

COX1 Functional Assays

The identification of COX1sv1 as a splice variant of COX1 provides a means for screening for compounds that bind to COX1sv1 protein thereby altering the ability of the COX1sv1 polypeptide to act as a dioxygenase or a peroxidase. Assays involving a functional COX1sv1 polypeptide can be employed for different purposes such as selecting for compounds active at COX1sv1, evaluating the ability of a compound to effect dioxygenase or peroxidase activity of COX1sv1, and mapping the activity of different COX1sv1 regions. COX1sv1 activity can be measured using different techniques such as: detecting a change in the intracellular conformation of COX1sv1; detecting a change in the intracellular location of COX1sv1; measuring the affinity of arachidonic acid substrate that binds to COX1sv1; measuring the kinetics of prostaglandin E2 synthesis in the presence of arachidonic acid and COX1sv1; or otherwise measuring other characteristics of dioxygenase or peroxidase activity of COX1sv1.

Recombinantly expressed COX1sv1 can be used to facilitate determining whether a compound is active at COX1sv1. For example, COX1sv1 can be expressed by an expression vector in a cell line and used in a co-culture growth assay, such as described in WO 99/59037, to identify compounds that bind to COX1sv1.

Techniques for measuring prostaglandin-endoperoxide synthase activity of COX1 are well known in the art (Hla and Neilson, 1992 Proc. Nat. Acad. Sci., 89:7384-7388; Vane et al., 1994 Proc. Nat. Acad. Sci., 91:2046-2050; Landino et al, 1997 J. Biol. Chem., 272:21565-21574; Smith et al., 1998 Proc. Nat. Acad. Sci., 95:13313-13318; Lu et al., 1999 J. Biol. Chem., 274:16162-16167). A large variety of other assays have been used to investigate the properties of COX1 and COX2 and therefore would also be applicable to the measurement of COX1sv1 function (for recent reviews see, Smith et al., 2000 Annu. Rev. Biochem., 69:145-82; Marnett et al., 1999 J. Biol. Chem., 274:22903-22906).

COX1sv1 functional assays can be performed using cells expressing COX1sv1 at a high level contacted with individual compounds or preparations containing different compounds (see, for example, WO 94/14977). A preparation containing different compounds where one or more compounds affect COX1sv1 in cells over producing COX1sv1 as compared to control cells containing expression vector lacking COX1sv1 coding sequence, can be divided into smaller groups of compounds to identify the compound(s) affecting COX1sv1 activity.

COX1sv1 functional assays can be performed using recombinantly produced COX1sv1 present in different environments. Such environments include, for example, cell extracts and purified cell extracts containing COX1sv1 expressed from recombinant nucleic acid and an appropriate membrane for the polypeptide; and the use of a purified COX1sv1 produced by recombinant means that is introduced into a different environment suitable for measuring cyclooxygenase activity.

Modulating COX1sv1 Expression

COX1sv1 expression can be modulated as a means for increasing or decreasing COX1sv1 activity. Such modulation includes inhibiting the activity of nucleic acids encoding the COX1 isoform target to reduce COX1 isoform protein or polypeptide expressions, or supplying COX1 nucleic acids to increase the level of expression of the COX1 target polypeptide thereby increasing COX1 activity.

Inhibition of COX1sv1 Activity

COX1sv1 nucleic acid activity can be inhibited using nucleic acids recognizing COX1sv1 nucleic acid and affecting the ability of such nucleic acid to be transcribed or translated. Inhibition of COX1sv1 nucleic acid activity can be used, for example, in target validation studies.

A preferred target for inhibiting COX1sv1 is mRNA translation. The ability of COX1sv1 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNAi also can be used to prevent protein expression of a target transcript. This method is based on the interfering properties of double-stranded RNA derived from the coding regions of gene that disrupts the synthesis of protein from transcribed RNA.

Enzymatic nucleic acid can recognize and cleave another nucleic acid molecule. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain anti-sense activities such as the ability to be cleaved by RNAse H, and can effect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999 Cell 99:123-32; Fire, et al., 1998 Nature 391:806-11), plants (Hamilton and Baulcombe, 1999 Science 286:950-52), Drosophila (Hammond, et al., 2001 Science 293:1146-50; Misquitta and Patterson, 1999 Proc. Nat. Acad. Sci. 96:1451-56; Kennerdell and Carthew, 1998 Cell 95:1017-26), and mammalian cells (Bernstein, et al., 2001 Nature 409:363-6; Elbashir, et al., 2001 Nature 411:494-8).

Increasing COX1sv1 Expression

Nucleic acid coding for COX1sv1 can be used, for example, to cause an increase cyclooxygenase activity or to create a test system (e.g., a transgenic animal) for screening for compounds affecting COX1sv1 expression. Nucleic acids can be introduced and expressed in cells present in different environments.

Guidelines for pharmaceutical administration in general are provided in, for example, Remington's Pharmaceutical Sciences, 18^{th} Edition, supra, and Modem Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of COX1sv1

To identify variants of the “normal” splicing of the exon regions encoding COX1, a series of RT-PCR reactions were designed to assay all of the intron-exon and exon-exon junctions of the COX1 gene. PolyA purified mRNA isolated from 79 different human tissues was obtained from BD Biosciences Clontech (Palo Alto, Calif.), Biochain Institute, Inc. (Hayward, Calif.), and Ambion Inc. (Austin, Tex.). Primers were designed using a Primer3 program (Whitehead Institute for Biomedical Research, Cambridge, Mass.) to amplify, among other nucleotide regions, a nucleotide region that spans the sequence between exon 4 and exon 7. The COX1₄₋₇ primer set (exon 4 forward primer: 5′ GTTCTGGGAGTTTGTCAATGCCACCTT 3′ [SEQ ID NO 8] and exon 7 reverse primer: 5′ ATTGTCTCCATAAATGTGGCCGAGGTCT 3′ [SEQ ID NO 9]) was expected to amplify a PCR product of 418 base pairs (bps).

Twenty-five nanograms (ng) of polyA mRNA from each tissue was subjected to a one-step reverse transcription-PCR amplification protocol using the Qiagen, Inc. (Valencia, Calif.), One-Step RT-PCR kit, using the following conditions:

Cycling conditions were as follows:

50° C. for 30 minutes;

95° C. for 15 minutes;

35 cycles of:

94° C. for 30 seconds;

62.5° C. for 40 seconds;

72° C. for 1 minutes; then

72° C. for 10 minutes.

RT-PCR amplification products (amplicons) were size fractionated on a 2% agarose gel (data not shown). One major RT-PCR amplicon was obtained from most human mRNA samples using the COX1₄₋₇ primer set. However, in several samples a faint amplicon band of about 300 nucleotides was also discernible. The about 300 nucleotide band was purified with a Qiagen Gel Extraction Kit from the human ovary sample and then subjected to further amplification using the COX1₄₋₇ primer set. The purified about DNA product was cloned using the Invitrogen TA cloning system (Gibco/Invitrogen, Carlsbad, Calif.). Transformants were screened using the same COX1₄₋₇ primer set to identify plasmids containing the about 300 nucleotide amplicon product. DNA from several clones having inserts of the expected size were then sent to Genome Therapeutics Corporation (Waltham, Mass.) for nucleotide sequencing of the cloned inserts. Analysis of the sequences obtained from the about 300 nucleotide clone insert DNAs revealed that the amplicon products consisted of 274 bps corresponding to 62 nucleotides at the 3′ end of exon 4, then 182 nucleotides of exon 6, and then 30 nucleotides at the 5′ end of exon 7. That is, the amplicon product corresponded to a precise drop of the exon 5 sequence found in the reference COX1 transcript, NM_{—}0080591, wherein the 3′ end of exon 4 was spliced to the 5′ end of exon 6. The detected alternative COX1 splice variant was designated as COX1sv1.

Table 1 presents a summary of the presence or absence of the about 274 nucleotide COXsv1 splice variant amplicon in 79 human tissue or cell line samples and a monkey brain sample.

| Sample | COX1 | COX1sv1 |
|---|---|---|
| Heart | + | − |
| Heart- aorta | + | − |
| Heart- atrioventrivcular nodes | + | − |
| Heart- interventricular septum | + | − |
| Heat- fetal | + | − |
| Tongue | + | − |
| Tonsil | + | − |
| Salivary Gland | + | − |
| Trachea | + | − |
| Stomach | + | − |
| Small intestine | + | − |
| Pancreas | + | − |
| Duodenum | + | − |
| Jejunum | + | − |
| Ileum | + | − |
| Ileocecum | + | − |
| Transverse colon | + | − |
| Descending colon | + | − |
| Rectum | + | − |
| Kidney | + | − |
| Kidney- fetal | + | − |
| Liver | + | − |
| Liver- fetal | + | − |
| Liver, left lobe | + | − |
| Bladder | + | − |
| Adrenal gland | + | − |
| Adrenal cortex | + | − |
| Adrenal medulla | + | − |
| Thyroid | + | − |
| Prostate | + | − |
| Testes | + | |
| Epididymus | + | |
| Uterus | + | |
| Uterus- corpus | + | |
| Placenta | + | − |
| Ovary | + | + (faint) |
| Spleen | + | + (faint) |
| Thymus | + | + (faint) |
| Lymph Node | + | + (faint) |
| Peripheral Leukocytes | + | − |
| Bone Marrow | + | − |
| Lung | + | − |
| Lung- fetal | + | − |
| Lung- upper right lobe | + | − |
| Adipose | + | − |
| Retina | + | − |
| Muscle- skeletal | + | − |
| Muscle- skeletal, fetal | + | − |
| Vertebra- fetal | + | − |
| HeLa (S3) | + (faint) | − |
| Leukemia Promyelocytic (HL-60) | + | − |
| Burkitts Lymphoma (Daudi) | + | − |
| Leukemia Chronic Myelogenous (K562) | + | − |
| Colorectal Adenocarcinoma (SW480) | + | − |
| Burkitts Lymphoma (Raji) | + | − |
| Melanoma (G361) | + | − |
| Lung Carcinoma (A549) | + | − |
| Brain | + | − |
| Brain-fetal | + | − |
| Brain- amygdale | + | − |
| Brain- caudate nucleus | + | − |
| Brain- corpus callosum | + | − |
| Brain- thalamus | + | − |
| Brain- cerebellum | + | − |
| Brain- cerebral cortex | + | − |
| Brain- hippocampus | + | − |
| Brain- postcentral gyrus | + | − |
| Brain- frontal lobe | + | − |
| Brain- medulla oblongata | + | − |
| Brain- occipital lobe | + | + (faint) |
| Brain- pariental lobe | + | − |
| Brain- pons | + | − |
| Brain- putamen | + | − |
| Brain- temporal lobe | + | − |
| Brain- hypothalamus | + | − |
| Brain- nucleus accumbens | + | − |
| Brain- paracentral gyrus | + | − |
| Spinal Chord | + | − |
| Spinal Cord- fetal | + | − |
| Monkey Brain | + | − |

As shown in Table 1, samples exhibiting both the about 418 base pair (NM_{—}080581) and the 274 base pair (COX1sv1) amplicons included ovary, spleen, thymus, lymph node, and occipital lobe of brain. For many other tissues a very faint band could also be seen at the about 274 base pair size range, indicating that the COX1sv1 splice variant may be present at very low levels in even more tissues then those listed in Table 1. All of the tissues and cell lines tested exhibited the 418 base pair form characteristic of COX1 (NM_{—}080581).

### Example 2

### Cloning of COX1sv1

RT-PCR data indicate that in addition to the normal reference COX1 mRNA sequence, NM_{—}080591 (encoding COX1 protein, NP_{—}542158), a splice variant form of COX1 mRNA (COX1sv1) also exists in some tissues. A full length COX1 clone having a nucleotide sequence comprising COX1sv1 as identified in Example 2 is isolated using a 5′ “forward” COX1 primer and a 3′ “reverse” COX1 primer, to amplify and clone the entire COX1sv1 mRNA coding sequence. The 5′ “forward” COX1 primer is designed to have a nucleotide of 5′ ATGAGCCCGAGTCTCTTGCTCTGGTTCTTGCT 3′ [SEQ ID NO 10]. The 3′ “reverse” COX1 primer is designed to have the nucleotide sequence of 5′ GAGCTCTGTGGATGGTCGCTCCACAGCACC 3′ [SEQ ID NO 11].

### RT-PCR

The COX1sv1 cDNA sequence is cloned using a combination of reverse transcription (RT) and polymerase chain reaction (PCR). More specifically, about 25 ng of testes polyA mRNA (Ambion, Austin, Tex.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clonetech (Palo Alto, Calif.) Advantage 2 PCR kit. PCR is done in a Gene Amp PCR System 9700 (Applied Biosystems, Foster City, Calif.) using the COX1 “forward” and “reverse” primers. After an initial 94° C. denaturation of 1 minute, 35 cycles of amplification are performed using a 30 second denaturation at 94° C. followed by a 1 minute annealing at 65° C. and a 90 second synthesis at 68° C. The 35 cycles of PCR are followed by a 7 minute extension at 68° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determined if the PCR has yielded products of the expected size, in the case of the predicted COX1sv1 mRNA, a product of about 1.5 kilobases (Kb). The remainder of the 50 μl PCR reactions from testes is purified using the QIAquik Gel extraction Kit (Qiagen, Valencia, Calif.) following the QIAquik PCR Purification Protocol provided with the kit. An about 50 μl of product obtained from the purification protocol is concentrated to about 6 μl by drying in a Speed Vac Plus (SC110A, from Savant, Holbrook, N.Y.) attached to a Universal Vacuum Sytem 400 (also from Savant) for about 30 minutes on medium heat.

### Cloning of RT-PCR Products

About 4 μl of the 6 μl of purified COX1sv1 RT-PCR product from testes is used in a cloning reaction using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative COX1sv1 clones are identified and prepared for a PCR reaction to confirm the presence of the polynucleotide amplicon expected to result from the splicing of the 3′ end of COX1 exon 4 to exon 6 of COX1. A 25 μl PCR reaction is performed as described above (RT-PCR section) to detect the presence of COX1sv1, except that the reaction includes miniprep DNA from the TOPO TA/COX1 ligation as a template, and uses the COX1₄₋₇ primer set. About 10 μl of each 25 μl PCR reaction is run on a 1% Agarose gel and the DNA bands generated by the PCR reaction are visualized and photographed on a UV light box to determine which minipreps samples have a PCR product of the size predicted for the predicted corresponding COX1sv1 splice variant mRNA. Clones having the COX1sv1 structure are identified based upon amplification of an amplicon band of 274 base pairs, whereas a normal reference COX1 clone will give rise to an amplicon band of 418 base pairs. DNA sequence analysis of the COX1sv1 cloned DNA produces a polynucleotide sequence having a COX1sv1 coding sequence of SEQ ID NO 1.

SEQ ID NO 1 contains an open reading frame that encodes a COX1sv1 protein (SEQ ID NO 2) identical to the reference protein COX1 (NP_{—}542158), but lacking the 48 amino acids encoded by exon 5 of the full length coding sequence of the reference COX1 mRNA (NM_{—}080591), except that the valine at amino acid 118 of the reference COX1 has been changed to a glycine due to creation of a new amino acid codon upon splicing of the 3′ end of the exon 4 nucleotide sequence to the 5′ end of the exon 6 nucleotide sequence of COX1 mRNA.

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 1, or the complement thereof.

2. The purified nucleic acid of claim 1, wherein said nucleic acid comprises a region encoding SEQ ID NO 2.

3. The purified nucleic acid of claim 1, wherein said nucleotide sequence encodes a polypeptide consisting of SEQ ID NO 2.

4. A purified polypeptide comprising SEQ ID NO 2.

5. The polypeptide of claim 4, wherein said polypeptide consists of SEQ ID NO2.

6. An expression vector comprising a nucleotide sequence encoding SEQ ID NO 2, wherein said nucleotide sequence is transcriptionally coupled to an exogenous promoter.

7. The expression vector of claim 6, wherein said nucleotide sequence encodes a polypeptide consisting of SEQ ID NO 2.

8. The expression vector of claim 6, wherein said nucleotide sequence comprises SEQ ID NO 1.

9. The expression vector of claim 6, wherein said nucleotide sequence consists of SEQ ID NO 1.

10. A method of screening for compounds able to bind selectively to COX1sv1 comprising the steps of:
(a) providing COX1sv1 polypeptide comprising SEQ ID NO 2;
(b) providing one or more COX1 or COX2 isoform polypeptides that are not COX1sv1,
(c) contacting said COX1sv1 polypeptide and said COX1 or COX2 isoform polypeptide that is not COX1sv1 with a test preparation comprising one or more compounds; and
(d) determining the binding of said test preparation to said COX1sv1 polypeptide and to said COX1 or COX2 isoform polypeptide that is not COX1sv1, wherein a test preparation that binds to said COX1sv1 polypeptide but does not bind to said COX1 or COX2 polypeptide that is not COX1sv1 contains a compound that selectively binds said COX1sv1 polypeptide.

11. The method of claim 10, wherein said COX1sv1 polypeptide is obtained by expression of said polypeptide from an expression vector comprising a polynucleotide encoding SEQ ID NO 2.

12. The method of claim 10, wherein said polypeptide consists of SEQ ID NO 2.

13. A method for screening for a compound able to bind to or interact with a COX1sv1 protein or a fragment thereof comprising the steps of:
(a) expressing a COX1sv1 polypeptide comprising SEQ ID NO 2 or fragment thereof from a recombinant nucleic acid;
(b) providing to said polypeptide a labeled COX1 ligand that binds to said polypeptide and a test preparation comprising one or more compounds; and
(c) measuring the effect of said test preparation on binding of said labeled COX1 ligand to said polypeptide, wherein a test preparation that alters the binding of said labeled COX1 ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are preformed using a whole cell.

16. The method of claim 13, wherein said polypeptide is expressed from an expression vector.

17. The method of claim 13, wherein said COX1sv1 ligand is a cyclooxygenase inhibitor.

18. The method of claim 16, wherein said expression vector comprises SEQ ID NO 1 or a fragment of SEQ ID NO 1.

19. The method of claim 13, wherein said polypeptide consists of the amino acid sequence of SEQ ID NO 2 or a fragment of SEQ ID NO 2.

20. The method of claim 13, wherein said test preparation contains one compound.


SEQUENCE LISTING (11 sequences)

SEQ ID NO: 1 - DNA, 1653 bp, Homo sapiens
atgagccggagtctcttgctctggttcttgctgttcctgctcctgctcccgccgctccccgtcctgctcgcggacccaggggcgcccacgccagtgaatccctgttgttactatccatgccagcaccagggcatctgtgtccgcttcggccttgaccgctaccagtgtgactgcacccgcacgggctattccggccccaactgcaccatccctggcctgtggacctggctccggaattcactgcggcccagcccctctttcacccacttcctgctcactcacgggcgctggttctgggagtttgtcaatgccaccttcatccgagagatgctcatgcgcctggtactcacagggaagaagcagttgccagatgcccagctcctggcccgccgcttcctgctcaggaggaagttcatacctgacccccaaggcaccaacctcatgtttgccttctttgcacaacacttcacccaccagttcttcaaaacttctggcaagatgggtcctggcttcaccaaggccttgggccatggggtagacctcggccacatttatggagacaatctggagcgtcagtatcaactgcggctctttaaggatgggaaactcaagtaccaggtgctggatggagaaatgtacccgccctcggtagaagaggcgcctgtgttgatgcactacccccgaggcatcccgccccagagccagatggctgtgggccaggaggtgtttgggctgcttcctgggctcatgctgtatgccacgctctggctacgtgagcacaaccgtgtgtgtgacctgctgaaggctgagcaccccacctggggcgatgagcagcttttccagacgacccgcctcatcctcataggggagaccatcaagattgtcatcgaggagtacgtgcagcagctgagtggctatttcctgcagctgaaatttgacccagagctgctgttcggtgtccagttccaataccgcaaccgcattgccatggagttcaaccatctctaccactggcaccccctcatgcctgactccttcaaggtgggctcccaggagtacagctacgagcagttcttgttcaacacctccatgttggtggactatggggttgaggccctggtggatgccttctctcgccagattgctggccggatcggtgggggcaggaacatggaccaccacatcctgcatgtggctgtggatgtcatcagggagtctcgggagatgcggctgcagcccttcaatgagtaccgcaagaggtttggcatgaaaccctacacctccttccaggagctcgtaggagagaaggagatggcagcagagttggaggaattgtatggagacattgatgcgttggagttctaccctggactgcttcttgaaaagtgccatccaaactctatctttggggagagtatgatagagattggggctcccttttccctcaagggtctcctagggaatcccatctgttctccggagtactggaagccgagcacatttggcggcgaggtgggctttaacattgtcaagacggccacactgaagaagctggtctgcctcaacaccaagacctgtccctacgtttccttccgtgtgccggatgccagtcaggatgatgggcctgctgtggagcgaccatccacagagctc

SEQ ID NO: 2 - protein, 551 aa, Homo sapiens
MSRSLLLWFLLFLLLLPPLPVLLADPGAPTPVNPCCYYPCQHQGICVRFGLDRYQCDCTRTGYSGPNCTIPGLWTWLRNSLRPSPSFTHFLLTHGRWFWEFVNATFIREMLMRLVLTGKKQLPDAQLLARRFLLRRKFIPDPQGTNLMFAFFAQHFTHQFFKTSGKMGPGFTKALGHGVDLGHIYGDNLERQYQLRLFKDGKLKYQVLDGEMYPPSVEEAPVLMHYPRGIPPQSQMAVGQEVFGLLPGLMLYATLWLREHNRVCDLLKAEHPTWGDEQLFQTTRLILIGETIKIVIEEYVQQLSGYFLQLKFDPELLFGVQFQYRNRIAMEFNHLYHWHPLMPDSFKVGSQEYSYEQFLFNTSMLVDYGVEALVDAFSRQIAGRIGGGRNMDHHILHVAVDVIRESREMRLQPFNEYRKRFGMKPYTSFQELVGEKEMAAELEELYGDIDALEFYPGLLLEKCHPNSIFGESMIEIGAPFSLKGLLGNPICSPEYWKPSTFGGEVGFNIVKTATLKKLVCLNTKTCPYVSFRVPDASQDDGPAVERPSTEL

SEQ ID NO: 3 - DNA, 40 bp, Homo sapiens
catgcgcctggtactcacagtgcgctccaaccttatcccc

SEQ ID NO: 4 - DNA, 40 bp, Homo sapiens
cacacccatgggaaccaaagggaagaagcagttgccagat

SEQ ID NO: 5 - DNA, 40 bp, Homo sapiens
catgcgcctggtactcacagggaagaagcagttgccagat

SEQ ID NO: 6 - DNA, 20 bp, Homo sapiens
gtactcacagggaagaagca

SEQ ID NO: 7 - protein, 10 aa, Homo sapiens
RLVLTGKKQL

SEQ ID NO: 8 - DNA, 27 bp, Homo sapiens
gttctgggagtttgtcaatgccacctt

SEQ ID NO: 9 - DNA, 28 bp, Homo sapiens
attgtctccataaatgtggccgaggtct

SEQ ID NO: 10 - DNA, 32 bp, Homo sapiens
atgagcccgagtctcttgctctggttcttgct

SEQ ID NO: 11 - DNA, 30 bp, Homo sapiens
gagctctgtggatggtcgctccacagcacc